(19) United States
(12) Patent Application Publication
(10) Pub. No.: US 2021/0164963 A1
(43) Pub. Date: Jun. 3, 2021

(54) DIAGNOSTIC STRIP AND DIAGNOSTIC SYSTEM USING THE SAME

(71) Applicant: QSTAG CO., LTD., Suwon-si Gyeonggi-do (KR)

(72) Inventors: Dong Hoon LEE, Seoul (KR); Da Woon HAN, Suwon-si , Gyeonggi-do (KR); Gyu Min KIM, Cheongju-si, Chungcheongbuk-do (KR)

(73) Assignee: QSTAG CO., LTD., Suwon-si Gyeonggi-do (KR)

(21) Appl. No.: 16/067,550

(22) PCT Filed: Feb. 7, 2018
(86) PCT No.: PCT/KR2018/001656

§ 371 Date: Jun. 29, 2018

(30) Foreign Application Priority Data
Mar. 24, 2017 (KR) 10-2017-0037711

(51) Int. Cl. G01N 33/487; G01N 33/563; G01N 33/574

(52) U.S. Cl. CPC G01N 33/48785; G01N 33/57484; G01N 33/563

(57) ABSTRACT

A diagnostic strip for reacting with a target for diagnosis, including: a connector configured to be connected to a power providing unit and provide a driving power to the diagnostic strip; an entry path configured to transfer a body fluid including the target; a reaction unit configured to react with the target and whose electrical characteristic is changed by the reaction; and a display unit configured to receive the driving power and whose display state is changed according to the changed electrical characteristic of the reaction unit.

### TECHNICAL FIELD

The present invention relates to a diagnostic strip and a diagnostic system using the same.

### BACKGROUND ART

Since existing test strips are only strip products, a collected body fluid such as blood is applied to a test strip, and a separate measuring instrument analyzes the composition of blood by using the test strip. Existing test strips only serve as carriers which provide a body fluid such as blood applied thereto to a separate measuring instrument, or existing test strips only provide inexact analysis results. Therefore, in some cases, such as when a body fluid is contaminated with an external material before carried and provided to a measuring instrument, it is not possible to ensure accuracy of measurement.

### DISCLOSURE

### Technical Problem

The present invention is directed to providing a strip and a diagnostic system for quantitatively testing a body fluid by using a portable terminal immediately after collecting the body fluid and checking test results.

### Technical Solution

One aspect of the present invention provides a diagnostic strip for reacting with a target for diagnosis, the diagnostic strip including: a connector configured to be connected to a power providing unit and provide a driving power to the diagnostic strip; an entry path configured to transfer a body fluid including the target; a reaction unit configured to react with the target and whose electrical characteristic is changed by the reaction; and a display unit configured to receive the driving power and whose display state is changed according to the changed electrical characteristic of the reaction unit.

Another aspect of the present invention provides a diagnostic system including: a diagnostic strip including a reaction unit whose electrical characteristic is changed by a reaction to a target included in a body fluid, a display unit configured to display a code whose display state is changed to correspond to the changed electrical characteristic, and a connector configured to be electrically connected to a power providing unit and receive a driving power; and the power providing unit configured to be electrically connected to the diagnostic strip through the connector and provide the driving power to the diagnostic strip.

### Advantageous Effects

According to the diagnostic strip and the diagnostic system, a body fluid is put in an entry path, and electric power is provided by a power providing unit such that a detection result can be immediately checked.

### DESCRIPTION OF DRAWINGS

FIG. 1 is a block diagram showing an overview of a diagnostic strip according to an exemplary embodiment.

FIG. 2 is a block diagram showing an overview of a diagnostic system according to an exemplary embodiment.

FIG. 3 is an exploded perspective view showing an overview of a diagnostic strip according to an exemplary embodiment.

FIG. 4 is a top-down transparent view showing an overview of a diagnostic strip according to an exemplary embodiment.

FIG. 5 is a flowchart schematically illustrating operation of a diagnostic system.

FIGS. 6 and 7 are perspective views illustrating operation of a diagnostic system.

FIG. 8 is a schematic circuit diagram of a reaction unit and a display unit.

FIG. 9 is a diagram schematically showing a diagnostic strip including a display unit and an overview display unit.

FIG. 10 is a perspective view showing an overview of a diagnostic strip.

FIG. 11 is a cross-section view schematically illustrating a diagnostic strip.

FIG. 12 is a block diagram illustrating operation of a diagnostic strip.

FIG. 13 is a schematic diagram showing an overview of a display unit.

FIGS. 14A and 14B are flowcharts illustrating operation of a diagnostic system.

FIG. 15 is a diagram showing a state in which a smart strip is connected to a portable terminal, which is a power providing unit.

### MODES OF THE INVENTION

### First Embodiment

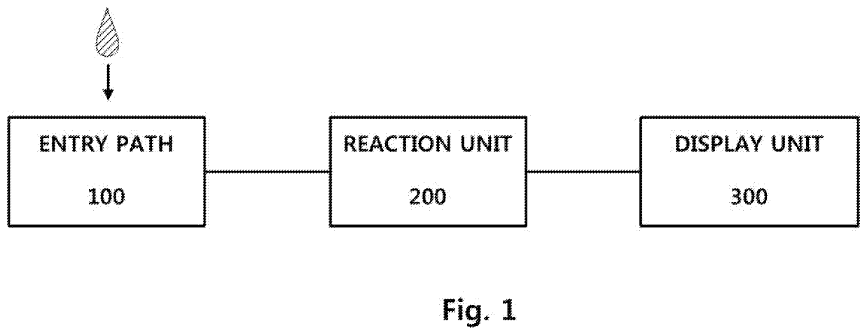

Hereinafter, a diagnostic strip according to an exemplary embodiment and a diagnostic system 1 using the same will be described with reference to the accompanying drawings. FIG. 1 is a block diagram showing an overview of a diagnostic strip 10 according to an exemplary embodiment. Referring to FIG. 1, the diagnostic strip 10 according to an exemplary embodiment includes an entry path 100 to which a body fluid including a target is provided, a reaction unit 200 which reacts with the target and whose electrical characteristic is changed by the reaction, and a display unit 300 whose display state is changed to correspond to the electrical characteristic changed by the reaction.

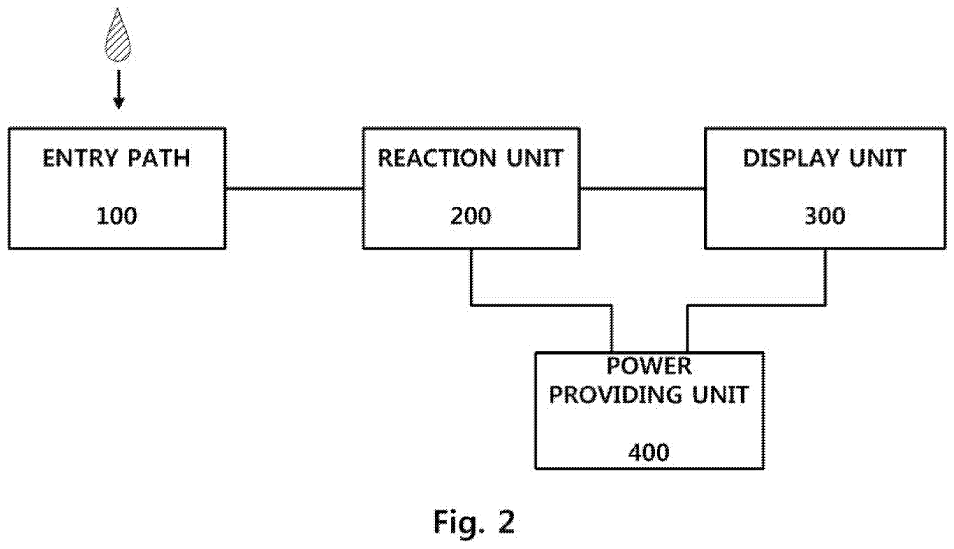

FIG. 2 is a block diagram showing an overview of a diagnostic system according to an exemplary embodiment. Referring to FIG. 2, a diagnostic system includes the diagnostic strip 10 including the reaction unit 200 whose electrical characteristic is changed by a reaction with the target included in a body fluid put through the entry path 100 and the display unit 300 displaying a code which can be read by a terminal 20 and whose display state is changed to correspond to the electrical characteristic changed by the reaction, and the diagnostic system also includes the terminal 20 for executing an application which provides a driving power to the strip 10 by driving the terminal 20, reads information from a code image acquired from the diagnostic strip 10, and controls the terminal 20 so that the information is displayed in the terminal.

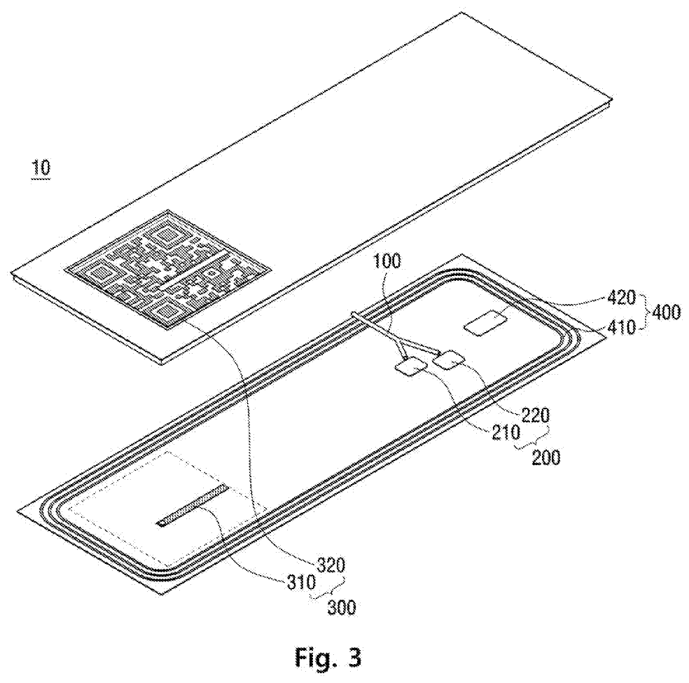

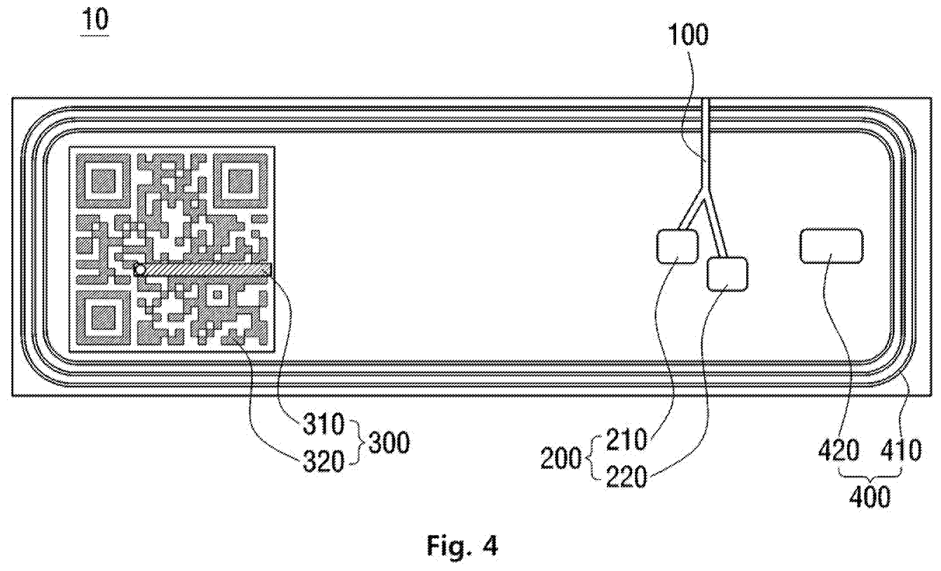

FIG. 3 is an exploded perspective view showing an overview of the diagnostic strip 10 according to an exemplary embodiment, and FIG. 4 is a top-down transparent view showing an overview of the diagnostic strip 10 according to an exemplary embodiment. Referring to FIGS. 3 and 4, the entry path 100 provides a body fluid to the reaction unit 200. The entry path 100 may have a shape for putting a body fluid in the diagnostic strip. As shown in the exemplary embodiments of FIGS. 3 and 4 as examples, the entry path 100 is formed as a capillary, and a body fluid is put in the reaction unit 200 in the diagnostic strip 10 due to the capillary action. According to an exemplary embodiment not shown in the drawings, the entry path 100 may be formed to expose the reaction unit 200, and a body fluid may be dropped on the reaction unit. According to another exemplary embodiment not shown in the drawings, the entry path may have a pipe form into which a tip of a carrying member, such as a syringe, including a body fluid is inserted.

The reaction unit 200 may include a first reaction unit 210 which reacts with a target for detection and a second reaction unit 220 which reacts with a body fluid. The first reaction unit 210 may further include a reactive material which reacts with the target for detection and whose electrical resistance value is changed. Except that the reactive material reacting with a target material is further formed in the first reaction unit 210, the first reaction unit 210 and the second reaction unit 220 may be formed of the same material or similar materials.

According to an exemplary embodiment, when the target for detection is glucose, the first reaction unit 210 may include, as the reactive material, a glucose oxidase whose electrical resistance value is changed by forming hydrogen peroxide (H₂O₂) as a result of an oxidation reaction with the glucose included in a body fluid. The first reaction unit 210 may react not only with the target in the body fluid but also with the body fluid such that an electrical characteristic may be changed. When the second reaction unit 220, which is identical or similar to the first reaction unit 210, is provided to calculate a difference between a reaction occurring at the first reaction unit 210 and a reaction occurring at the second reaction unit 220, it is possible to exclude a result of a reaction between the body fluid and the reaction unit and detect a change in electrical characteristic caused by a reaction between the target and the reactive material.

According to an exemplary embodiment, the reaction unit 200 may be formed by performing a printing process. As the printing process for forming the reaction unit, it is possible to use a transfer printing process in which printing is performed after a material for forming the reaction unit is applied to a mold, an inkjet printing process in which a material for forming the reaction unit is discharged from a nozzle, a gravure printing process in which a roller is used to print a material for forming the reaction unit, and a roll-to-roll printing process. Also, during the process of printing the reaction unit 200, one or more of the reaction unit 200, the display unit 300, and a power providing unit 400 may be formed.

In the exemplary embodiment shown in FIGS. 3 and 4, the display unit 300 may display a machine-readable code., Reaction information between the target and the reactive material is displayed as the code displayed by the display unit 300 according to predetermined encoding rules and can be read through a device.

According to an exemplary embodiment, the display unit 300 may include a variable code display unit 310 whose displayed information is changed by a reaction between a body fluid and the reaction unit 200 and a fixed code display unit 320 whose displayed information is not changed even when a reaction occurs between a body fluid and the reaction unit 200.

According to an exemplary embodiment, the code displayed by the display unit 300 is a quick response (QR) code, which is changed according to a reaction result and displays information resulting from the reaction. In an exemplary embodiment not shown in the drawings, a code displayed by the display unit is a barcode. In another exemplary embodiment not shown in the drawings, a code displayed by the display unit may be a display bar whose color can be changed according to a quantitative value.

In an exemplary embodiment, the variable code display unit 310 may include a light emitting device, such as a light-emitting diode (LED) or an organic light-emitting diode (OLED), which may or may not emit light due to a voltage provided by a reaction between the target and the reaction unit 200, thereby changing a code displayed by the variable code display unit 310.

In another exemplary embodiment, the variable code display unit 310 may include a variable color device. In an example, the variable color device may be implemented as a device in which colloid particles are distributed and locations of the colloid particles are changed by a voltage provided to the device such that a color displayed to the outside is changed.

In another example, the variable color device may be implemented as an electrochromic device. The electrochromic device denotes a device including an electrochromic material whose color is changed by an electrochemical oxidation-reduction reaction when a voltage is provided. The electrochromic device is changed in color when a voltage is provided.

In an example, the electrochromic device may include WO₃, Nb₂O₅, MoO₃, and TiO₃, which are cathodic coloration materials which become colorless in an oxidized state, as electrochromic materials. In another example, the electrochromic device may include V₂O₅, IrO₂, and NiO, which are anodic coloration materials displaying a color in an oxidized state, as electrochromic materials.

Therefore, the code displayed by the variable code display unit 310 is changed according to a voltage provided by a reaction between the target and the reaction unit 200.

In another exemplary embodiment, the variable code display unit 310 may be implemented as an electronic (e)-ink. E-ink includes a capsule and pigments which are injected into the capsule and charged with different electrical charges. When a voltage generated by a reaction between the target and the reaction unit 200 is provided between one side and another side of the capsule, the charged pigments move according to a level and a polarity of the voltage and change the code displayed by the variable code display unit 310.

The fixed code display unit 320 displays a determined code irrespective of a reaction between the target and the reactive material. Since the displayed content is not changed, the fixed code display unit 330 may be printed and displayed. As an example, when the code displayed by the display unit 300 is a QR code, the variable code display unit 310 encodes the reaction information according to QR code rules and displays the encoded reaction information, and the fixed code display unit 320 displays an element which indicates a directionality of the QR code. As another example, when the code displayed by the display unit 300 is a barcode, the fixed code display unit 320 may be a guard bar which is displayed on the left and right and at the center of the barcode so that the reaction information may be recognized. The variable code display unit 310 may be positioned in the fixed code display unit 320 and may encode the reaction information into a combination of bars having different thicknesses and display the encoded reaction information.

The power providing unit 400 provides power to the diagnostic strip 10. According to an exemplary embodiment, the power providing unit 400 may include an inductive coupling device, which is inductively coupled to the terminal and receives power. For example, the inductive coupling device may include a rectenna which collects and rectifies a radio wave provided by the terminal and outputs a rectified radio wave. The rectenna may include an antenna 410, which is formed as a coil to receive power wirelessly transmitted by the terminal 20, and a rectification device 420 which rectifies a received signal. In an exemplary embodiment not shown in the drawings, the power providing unit 400 may further include a capacitor which smooths a pulse wave output by the rectenna and stores energy. In an exemplary embodiment, as described above, the power providing unit 400 may be formed through a printing process for forming the reaction unit 200 and the display unit 300.

According to another exemplary embodiment, the power providing unit may include a button cell or a coin cell and enable a user to use the button cell or the coin cell together with the inductive coupling device.

According to another exemplary embodiment, the power providing unit may include an energy conversion device and an electric energy storage device. The energy conversion device is a device which converts provided mechanical energy into electrical energy. A user provides mechanical energy to the energy conversion device, and the energy conversion device converts the mechanical energy into electrical energy and outputs the electrical energy to the electric energy storage device. In an example, the energy conversion device may be a piezoelectric device which converts mechanical energy provided by a user pressing the energy conversion device into electrical energy. In another example, the energy conversion device may be a device which converts mechanical energy caused by friction into electrical energy.

The diagnostic strip 10 may be driven by using energy stored in the electric energy storage device.

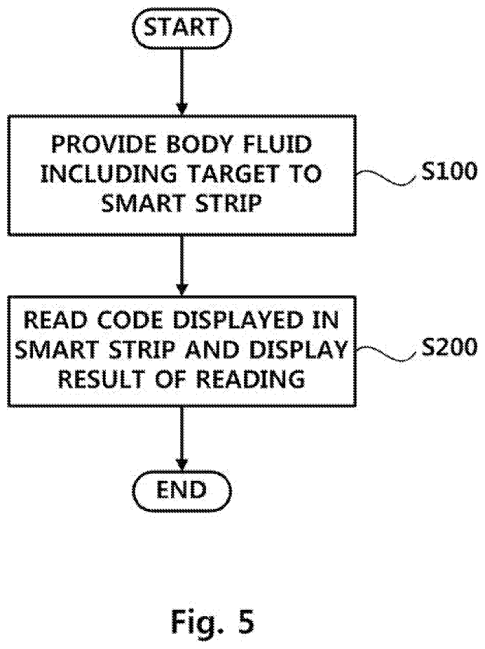

Operation of the diagnostic system 1 will be described below. FIG. 5 is a flowchart schematically illustrating operation of the diagnostic system 1. In an exemplary embodiment, a body fluid including a target is provided through the entry path 100 of the diagnostic strip 10 (S100). When the body fluid is provided, the first reaction unit 210, which includes a reactive material that reacts with the target, reacts with the body fluid and the target such that an electrical characteristic may be changed. The second reaction unit 220 provided with the body fluid reacts with the body fluid such that an electrical characteristic thereof may be changed.

In an exemplary embodiment, the terminal 20 may be inductively coupled to the diagnostic strip 10, thereby providing driving power to the diagnostic strip 10. In another exemplary embodiment, the diagnostic strip 10 may be provided with driving power from a primary cell embedded therein. A process of providing the driving power to the diagnostic strip 10 may be performed, in an example, after the process of providing a body fluid to the diagnostic strip 10, and in another example, before the process of providing a body fluid to the diagnostic strip 10.

The diagnostic strip 10 provided with the driving power may display a code corresponding to a reaction result on the display unit 300, and the terminal 20 may read the code and provide the reaction result to a user (S200). According to an exemplary embodiment, the display unit 300 may obtain a difference between a reaction result of the first reaction unit 210 and a reaction result of the second reaction unit 220 and generate a code corresponding to the difference, thereby displaying the code.

According to an exemplary embodiment, the terminal 20 may save the result of reading to a server through a communication network. According to another exemplary embodiment, the terminal 20 may store the result of reading therein. When it is necessary to monitor reaction results for a long time period, the terminal 20 may depict and provide the stored information to the user in the form of a graph.

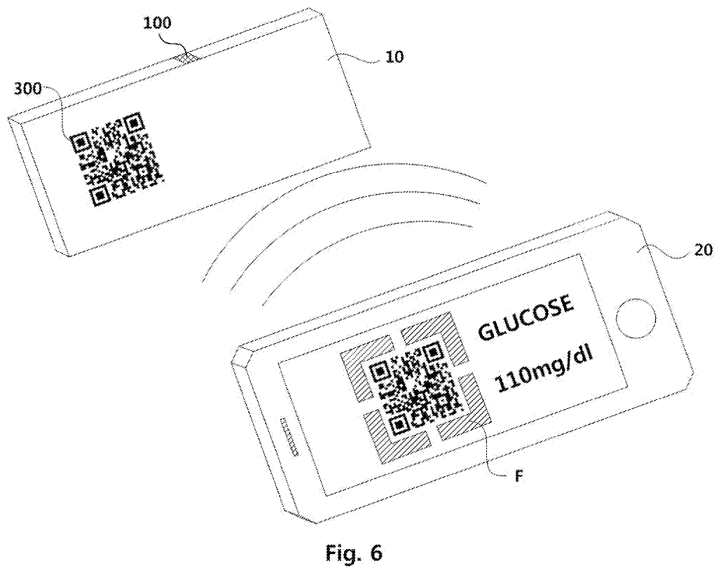

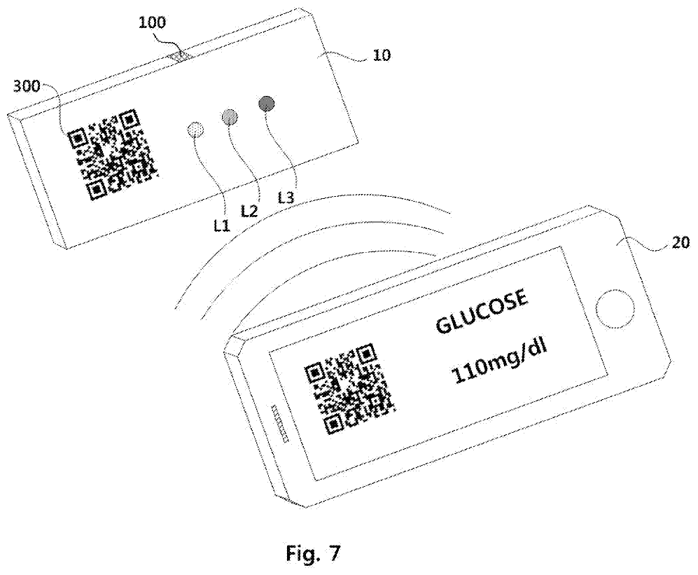

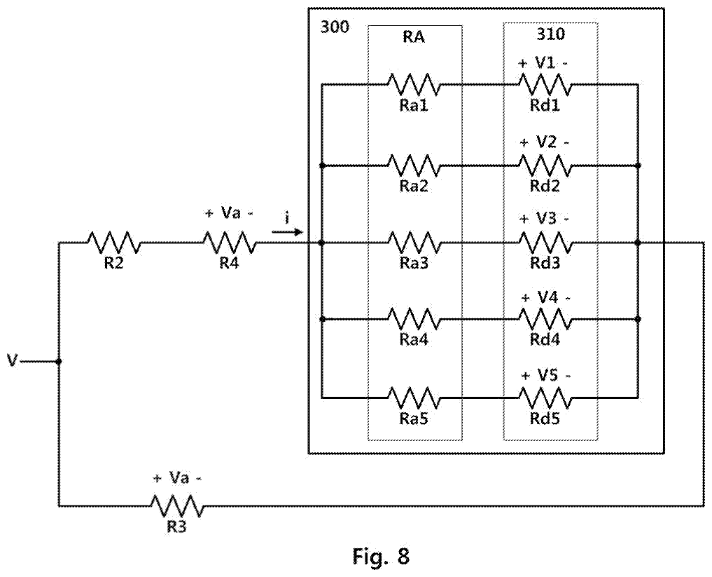

FIGS. 6 and 7 are perspective views illustrating operation of the diagnostic system 1, and FIG. 8 is a schematic circuit diagram of the reaction unit 200 and the display unit 300. Referring to FIGS. 6 and 7, when a body fluid is provided to the entry path 100, the body fluid is provided to the reaction unit 200. In an exemplary embodiment, when a user runs an application on the portable terminal 20, the application may control the portable terminal 20 so that a frame F and the display unit 300 of the diagnostic strip may be displayed on a screen of the portable terminal 20. According to an exemplary embodiment, the application may acquire an image of the display unit 300 of the diagnostic strip 10 by using a camera (not shown) included in the portable terminal and display the acquired image with the frame F on the screen of the portable terminal.

According to an exemplary embodiment, the application may provide power while a distance between the portable terminal 20 and the diagnostic strip 10 is maintained at a predetermined distance. When the distance between the portable terminal 20 and the diagnostic strip 10 is short, the portable terminal 20 may provide a voltage which is unnecessarily high to the diagnostic strip 10, and when the distance between the portable terminal 20 and the diagnostic strip 10 is long, the portable terminal 20 may not provide a voltage sufficient to drive the display unit 300.

In an example, as shown in FIG. 6, the frame F displayed on the screen of the portable terminal 20 may be compared with the display unit 300 in size, and when the frame F and the display unit 300 correspond to each other in size, power may be supplied to the portable terminal through the portable terminal.

In another example, as shown in FIG. 7, one or more light emitting devices L1, L2, and L3 may be provided in the diagnostic strip 10 to indicate a level of voltage provided by the portable terminal. As an example, in an exemplary embodiment in which one light emitting device is included, the light emitting device may be controlled to emit light only when the level of voltage provided by the portable terminal 20 is appropriate. As another example, in an exemplary embodiment in which a plurality of light emitting devices are included, any one light emitting device may emit light when the voltage provided by the portable terminal 20 is higher than an objective voltage, another light emitting device may emit light when the voltage provided by the portable terminal 20 is lower than the objective voltage, and another light emitting device may emit light when the voltage provided by the portable terminal 20 is within a range of the objective voltage. According to another exemplary embodiment not shown in the drawings, the level of voltage provided to the diagnostic strip 10 may be depicted in the form of text, color, barcode, or the like.

Therefore, the user may adjust the distance between the portable terminal 20 and the diagnostic strip 10 so that the portable terminal 10 may provide an appropriate driving voltage to the diagnostic strip 10.

According to another exemplary embodiment, the application may control the terminal to wirelessly transmit power when the user runs the application. According to an exemplary embodiment, the portable terminal 20 may provide power to the diagnostic strip 10 by using a method such as near field communication (NFC), radio frequency identification (RFID), and the like.

According to an exemplary embodiment, the power providing unit 300 may further include a Zener diode (not shown) which clamps a level of provided voltage. When the distance between the portable terminal 20 and the diagnostic strip 10 is short, a voltage equal to or higher than a voltage required for driving may be provided to the diagnostic strip 10. When a voltage equal to or higher than the voltage required for driving is provided, the Zener diode may clamp the voltage.

When the driving power is provided from the portable terminal 20 to the diagnostic strip 10, the display unit 300 may display a target detection result. When a user intends to detect the amount of a target in a body fluid, the reactive material, which is included in the first reaction unit 210 and reacts with the target, reacts with the target, and an electrical resistance value thereof is changed. Also, the first reaction unit 210 may react with a component other than the target, and the electrical resistance value may be changed. However, due to the second reaction unit 220, it is possible to compensate for influence of the reaction to the component other than the target.

In an exemplary embodiment for detecting glucose in blood, blood put in the entry path 100 may be provided to the first reaction unit 210 and the second reaction unit 220. A change in electrical characteristic exhibited by the first reaction unit 210 is the sum of a change made by a reaction between glucose in the blood and the first reaction unit 210 and a change made by a reaction between the blood excluding glucose in the blood and the first reaction unit 210. Since glucose oxidase which reacts with glucose is not included in the second reaction unit 210, the second reaction unit 220 may react with components of blood other than glucose such that an electrical characteristic thereof may be changed.

As shown in FIG. 8, it is possible to assume that an electrical resistance resulting from the first reaction unit 210 reacting with glucose is R2, an electrical resistance resulting from the first reaction unit 210 reacting with a body fluid other than a target is R4, and an electrical resistance resulting from the second reaction unit 220 reacting with the body fluid is R3. Also, a voltage V provided by the portable terminal 10 may be provided to one end of a closed loop shown in the drawing, and identical currents i may flow through R2, R4, and R3 because R2, R4, and R3 are included in the same closed loop. In this case, the first reaction unit 210 may be identical or similar to the second reaction unit 220 except that the first reaction unit 210 further includes a reactive material which reacts with the target and whose electrical characteristic is changed. Therefore, a voltage drop Vb occurring in the electrical resistance R3 resulting from the second reaction unit 220 reacting with the body fluid may have the same level as a voltage drop Va occurring in the electrical resistance R4 resulting from the first reaction unit 210 reacting with the body fluid other than the target. Consequently, Va and Vb may have similar levels and thus cancel each other out. A voltage provided to both ends of the display unit 300 results from a voltage drop caused by the reactive material reacting with the target in the body fluid.

The display unit 300 may include a resistor array RA. The resistor array RA includes resistors having different resistance values. In FIG. 8, display devices included in the variable code display unit 310 are modeled as resistors Rd1, Rd2, . . . , and Rd5 connected to resistors included in the resistor array RA. When a voltage is provided to the both ends of the display unit 300, the voltage is divided to correspond to a resistance value included in the resistor array and an equivalent resistance value of the variable code display unit.

Voltages provided to both ends of equivalent resistors of the variable code display unit 310 correspond to resistance ratios between the resistors included in the resistor array and the equivalent resistors of the variable code display unit 310. For example, Ra1, Ra2, Ra3, Ra4, and Ra5 included in the resistor array may be respectively 0.5Ω, 1Ω, 1.5Ω, 2Ω, and 2.5Ω, Rd1 to Rd5 are all 1Ω, voltages provided to the display devices modeled as Rd1 to Rd5 may be respectively V1, V2, . . . , and V5, and the display devices modeled as Rd1 to Rd5 may be turned on at a voltage of 1.3 V or above.

When a voltage provided to the display unit 300 according to a change in resistance made by a reaction between the target in the body fluid and the reactive material is 3 V, the both-end voltages V1 to V5 of Rd1 to Rd5 which are obtained through voltage dividing are approximately 2 V, 1.5 V, 1.2 V, 1 V, and 0.86 V, respectively. Since both V1 and V2 are equal to or high than the turn-on voltage, it is possible to see that the display devices modeled as Rd1 and Rd2 are turned on and the other display devices are turned off.

As another example, when the voltage provided to the display unit 300 is 6 V, the both-end voltages V1 to V5 of Rd1 to Rd5 which are obtained through voltage dividing are approximately 4 V, 3 V, 2.4 V, 2 V, and 1.71 V, respectively. Since all the both-end voltages V1 to V5 are equal to or high than the turn-on voltage, the display devices modeled as Rd1 to Rd5 may be turned on.

Therefore, the code displayed by the variable code display unit 310 may be changed by a voltage formed according to a change in resistance made by a reaction between the target and the reactive material, and the display unit 300 may display codes accordingly corresponding to a target concentration in the body fluid, whether the target exists in the body fluid, and the like.

The portable terminal 20 may read the codes displayed by the display unit 300 and display results of reading on the screen. Referring to FIG. 6, when it is intended to detect a glucose level in blood, the display unit 300 displays a code corresponding to a glucose level in blood, and the portable terminal 20 reads the code and displays a result of reading on the screen. According to an exemplary embodiment, the portable terminal may provide results of reading to the server so that personalized measurement results may be stored.

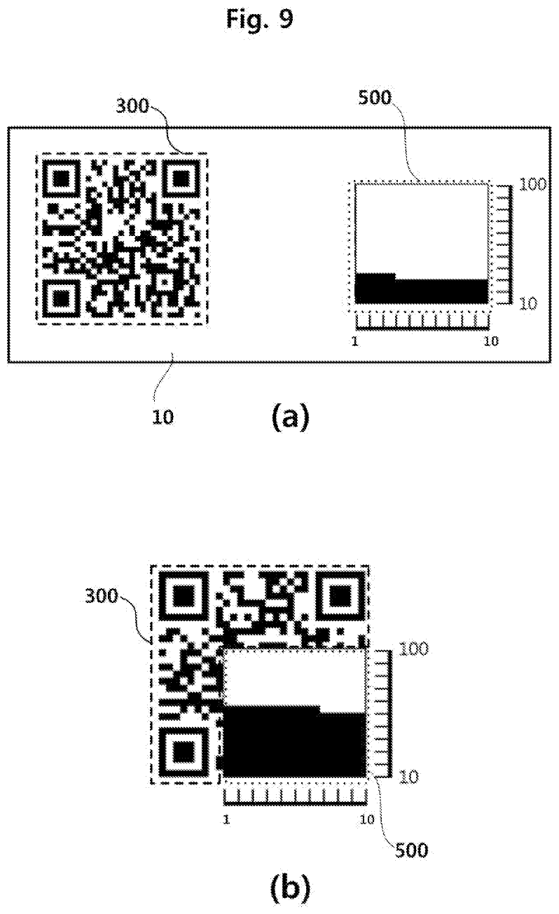

According to another exemplary embodiment of the diagnostic strip 10, the diagnostic strip 10 may further include an overview display unit which schematically displays a reaction result between the target and the reaction unit to a user. FIG. 9 is a diagram schematically showing the diagnostic strip 10 including the display unit 300 and an overview display unit 500. Referring to FIG. 9, as shown in an example in FIG. 9A, the display unit 300 and the overview display unit 500 may be disposed separately on a surface of the diagnostic strip 10. Ten pixels may be disposed horizontally in a line, and respective lines may be disposed vertically pixel by pixel. As shown in the example in the drawing, two lines in each of which 10 pixels are horizontally disposed are vertically disposed, and three pixels display a code in the uppermost line such that a code displayed by the variable code unit corresponds to 23. Therefore, it is possible to simply find a result of measuring by reading the code.

As shown in another example in FIG. 9B, the overview display unit 500 may be displayed to overlap the display unit 300. As an example, the overview display unit 500 may have 10 pixels horizontally disposed in a line and have respective lines vertically disposed in a neat pile, wherein a value corresponding to one pixel is 5. As shown in the example of the drawing, two lines in each of which 10 pixels are horizontally disposed are vertically disposed, and three pixels display a code in the uppermost line, such that a code displayed by the variable code unit corresponds to 23. Since a value corresponding to one pixel is 5, it is possible to see that the code corresponds to 115 by reading the code accordingly.

Like the variable code display unit 310, the overview display unit 500 may be implemented with a light emitting device, such as an LED, an OLED, etc., a variable color device, an electrochromic device, an e-ink, and the like whose display states are changed to correspond to an electrical characteristic which is changed by a reaction between the reaction unit and the target.

### Second Embodiment

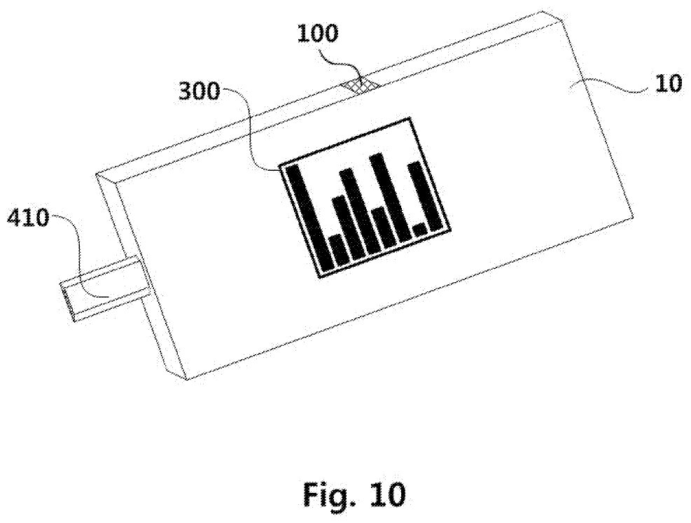

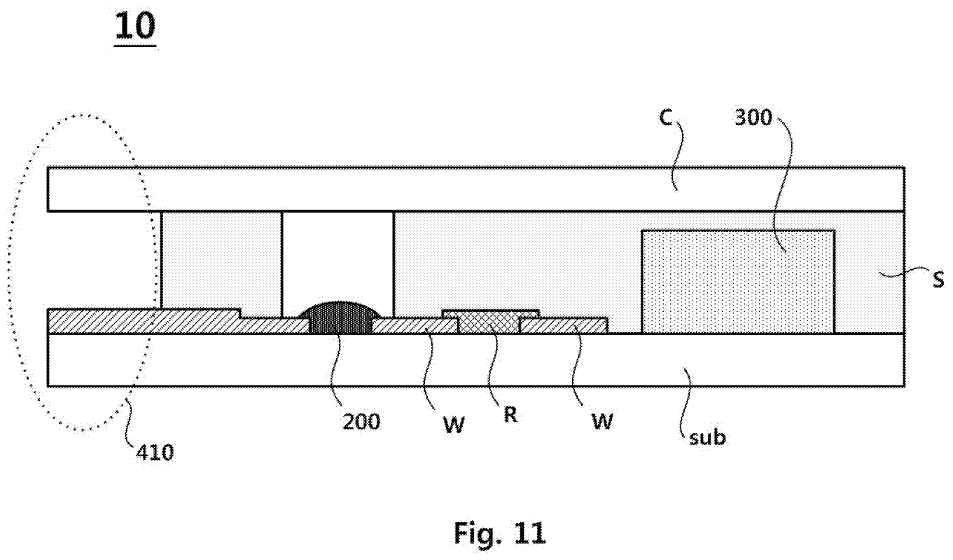

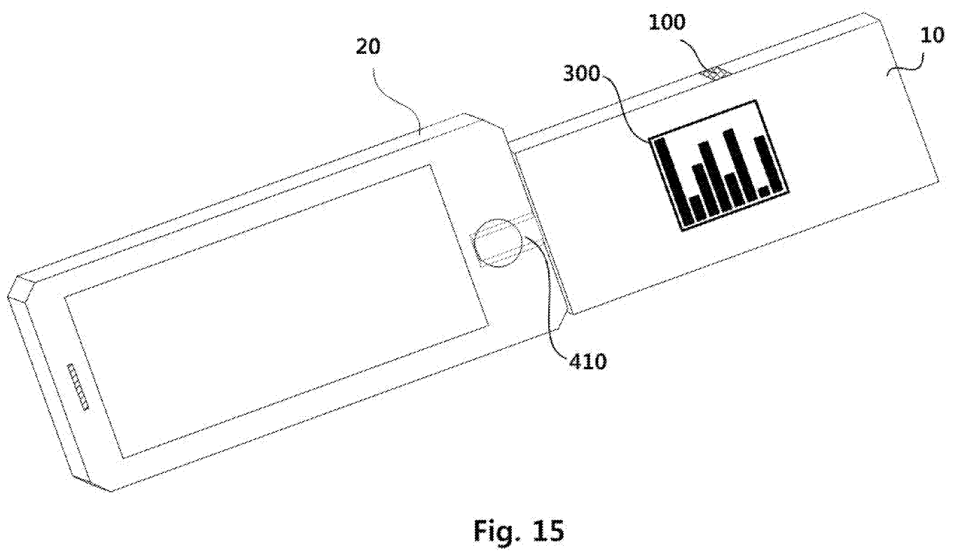

Hereinafter, an exemplary embodiment of a diagnostic strip and a diagnostic system using the diagnostic strip will be described. For brief and clear description, descriptions which are identical or similar to the above description may be omitted. FIG. 10 is a perspective view showing an overview of a diagnostic strip 10, and FIG. 11 is a cross-section view schematically illustrating the diagnostic strip 10. Referring to FIG. 10, the diagnostic strip 10 reacts with a target so that a diagnosis may be made. The diagnostic strip 10 includes a connector 410 which is connected to a power providing unit 20 (see FIG. 15) and provides a driving power from the power providing unit to the diagnostic strip, an entry path 100 which transfers a body fluid including the target to a reaction unit 200, the reaction unit 200 which reacts with the target and whose electrical characteristic is changed by the reaction, and a display unit 300 which is provided with the driving power and whose display state is changed according to the changed electrical characteristic of the reaction unit 200.

According to the exemplary embodiment shown in FIG. 10, the driving power for driving the diagnostic strip 10 is provided from the power providing unit 20 (see FIG. 15) through the connector 410. As an example, the power providing unit may be a mobile terminal, such as a cellular phone, a tablet, or the like, and as another example, the power providing unit may be a personal computer. As another example, the power providing unit may be an auxiliary battery.

As an exemplary embodiment, the connector may be a connector conforming to a universal serial bus (USB) standard. As shown in the example of FIG. 10, the connector may be a micro-B type 5-pin male connector of a USB standard. In another example not shown in the drawing, the connector may be any one of a micro-B type female connector, a standard A type male connector, a standard A type female connector, a standard B type male connector, a standard B type female connector, a mini B type male connector, a mini B type female connector, a C type male connector, and a C type female connector. In another example not shown in the drawing, the connector may be any one of a lightning male connector and a lightning female connector conforming to a lightning connector standard.

In an exemplary embodiment, when the connector is a male connector, the connector may be inserted into a female connector formed in the power providing unit and provided with power, and when the connector is a female connector, the connector may be provided with power through a cable connected to the power providing unit, or a male connector of the power providing unit may be inserted into the connector such that power may be provided.

FIG. 11 is a schematic cross-sectional view of the diagnostic strip 10. To facilitate in understanding, FIG. 11 exaggeratedly shows thickness and size. Referring to FIG. 11, a substrate sub may be a synthetic resin substrate. For example, the substrate sub may be formed of a synthetic resin such as polyethylene terephthalate (PET) and the like.

As shown in the drawing, a conductive material wire w is disposed on one surface of the substrate sub according to a connector standard, and the substrate sub and a cover C are cut according to the connector standard such that the connector 410 may be formed. For example, as the conductive material wire w, conductive paste including silver (Ag) may be printed with a printing technique, such as inkjet printing, gravure printing, transfer printing, or the like, according to the connector standard. As an example not shown in the drawing, a connector module conforming to an objective connector standard may be attached to a diagnostic strip.

As an exemplary embodiment, the display unit 300 may be an electrochromic device. As an example, the electrochromic device is formed by stacking a transparent electrode, an anodic coloration material layer or an ion storage layer, an electrolyte layer, a cathodic coloration material, and a transparent electrode between one pair of substrates. When a voltage is provided in one direction between the transparent electrodes, the anodic coloration material layer or the ion storage layer provides positive ions and thus are oxidized and colored, and the cathodic coloration material layer provided with the positive ions through the electrolyte layer are reduced and colored. When a voltage is provided in the reverse direction between the transparent electrodes, the oxidation and reduction reactions occur vice versa, and the anodic coloration material layer or the ion storage layer and the cathodic coloration material layer are decolorized and become transparent.

In an exemplary embodiment, the display unit 300 may be implemented in the form of a display bar as shown in the example of FIG. 10. The display bar may display surrounding information, such as whether there is the target to be detected, a concentration of the target, a hydrogen-ion concentration, a temperature, a humidity, etc., which has influence on target detection, to correspond to detected values. In an exemplary embodiment not shown in the drawing, the display unit 300 may display the display bar together with a machine-readable code, such as a QR code or a barcode, and the overview display unit shown in FIG. 9 as an example may be further displayed.

The reaction unit 200 is a material which reacts with the target to be detected and whose electrical characteristic is changed. In an exemplary embodiment, the reaction unit is a material whose electrical characteristic is changed by an enzyme reaction with the target for detection, or an oxidation-reduction reaction related to the enzyme reaction. For example, the reaction unit may include a glucose oxidizer which reacts with glucose, a cholesterol oxidizer which reacts with cholesterol, and the like.

In another exemplary embodiment, the reaction unit may be a material whose electrical characteristic is changed by an antigen-antibody reaction with the target for detection. For example, the reaction unit may be any one of an anti-influenza antibody which reacts with an avian influenza (AI) virus, anti-epithelial cell adhesion molecule (EpiCAM), anti-prostate-specific antigen (PSA), anti-human epidermal growth factor receptor 2 (HER2), anti-carcinoembryonic antigen (CEA), and anti-cancer antigen (CA) antibodies which react with cancer cells, and an anti-apolipoprotein B antibody which reacts with lipids in blood.

In another exemplary embodiment, the reaction unit may further include a probe which is complementarily bound with the target to be detected, and may be a material which binds to the target and whose electrical characteristic is changed. For example, the reaction unit may include a probe having an aptamer which binds to protein, which is the target for detection, and a nucleotide marker, and nucleotides having a sequence complementary to the target for detection.

According to an exemplary embodiment, the reaction unit 200 may be manufactured as an electrochemical sensor which uses a reaction to the composition of a specific enzyme and an oxidation-reduction reaction related to the reaction. According to another exemplary embodiment, the reaction unit 200 may be manufactured as a sensor which uses a binding reaction of a receptor.

For example, in a reaction in which an enzyme is used, an electrical change is detected through an oxidation-reduction reaction of an electron transfer mediator. Also, a receptor which selectively binds to viruses, protein, cancer cells, deoxyribonucleic acid (DNA), etc. is fixed through a specific chemical reaction, and an electrical change caused by the binding reaction with the target is sensed.

The receptor, such as the aptamer, and an antigen may be directly applied to a surface. As another example, graphene oxide, poly (3,4-ethylenedioxythiophene): polystyrene sulfonate (PEDOT:PSS), gold nanoparticles, etc. may be applied by using an auxiliary device. For example, the reaction unit 200 may be formed with a printing technique, such as inkjet printing, gravure printing, transfer printing, or the like.

As an example, the reaction unit 200 may be a material which reacts with glucose and whose electrical characteristic is changed. As another example, the reaction unit 200 may be a material which reacts with the AI virus and whose electrical characteristic is changed. As another example, the reaction unit 200 may be a material which reacts with cancer cells and whose electrical characteristic is changed. As another example, the reaction unit 200 may be a material which reacts with cholesterol and whose electrical characteristic is changed. As another example, the reaction unit 200 may be a material which reacts with lipids in blood and whose electrical characteristic is changed.

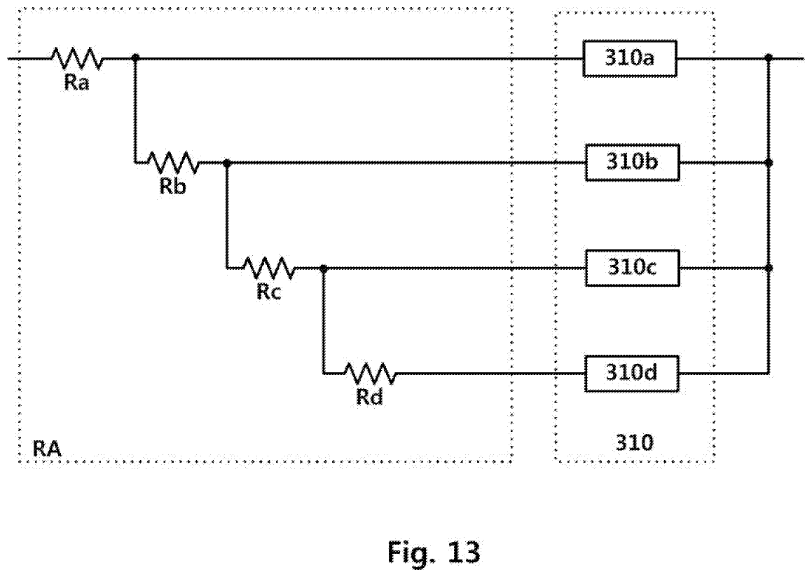

Resistors R are formed in an array and may be connected to unit display devices included in a variable code display unit 310 (see FIG. 13). The resistors may be formed of a material having a known resistivity with a preset length to have an objective resistance value. As an example, the resistors may be formed to have the objective resistance value by printing PEDOT:PSS with the preset length.

The cover C may be coupled to the substrate sub and protect the diagnostic strip 10. For example, the cover C may be coupled to the substrate sub in a coupling form, such as projection-recess coupling, screw coupling in which a screw is used, or coupling in which a barb-shaped hook is used, or the cover C and the substrate sub may be bonded to a spacer S and coupled to each other. The spacer S may maintain a distance between the cover C and the substrate sub and prevent an unnecessarily high pressure from being applied to a structure formed on the substrate upon coupling. For example, the cover C and the spacer S may be formed of a synthetic resin, which may be the same material as that of the substrate sub. According to an exemplary embodiment, the cover C is formed of a transparent material and thus transmits content displayed by the display unit 300. According to another exemplary embodiment, the cover C may be formed of an opaque material, but a window may be formed to transmit content displayed by the display unit 300.

In an exemplary embodiment, the conductive material wire w and the resistors R are printed and formed on the substrate sub, the pre-assembled display unit 300 is disposed in the substrate sub, the reaction unit 200 is printed, and the cover C is coupled to the substrate so that the diagnostic strip 10 may be formed. In another exemplary embodiment, a connector module is connected to the substrate sub in which the conductive material wire w, the resistors R, the display unit 300, and the reaction unit 200 are formed, and the cover C is coupled to the substrate so that the diagnostic strip 10 may be formed. In another exemplary embodiment, the conductive material wire w and the resistors R are printed and formed on the substrate sub, the display unit 300 is formed by stacking a transparent electrode, an anodic coloration material layer or an ion storage layer, an electrolyte layer, a cathodic coloration material, and a transparent electrode, and then the diagnostic strip 10 may be formed by printing the reaction unit 200 and coupling the cover C to the substrate.

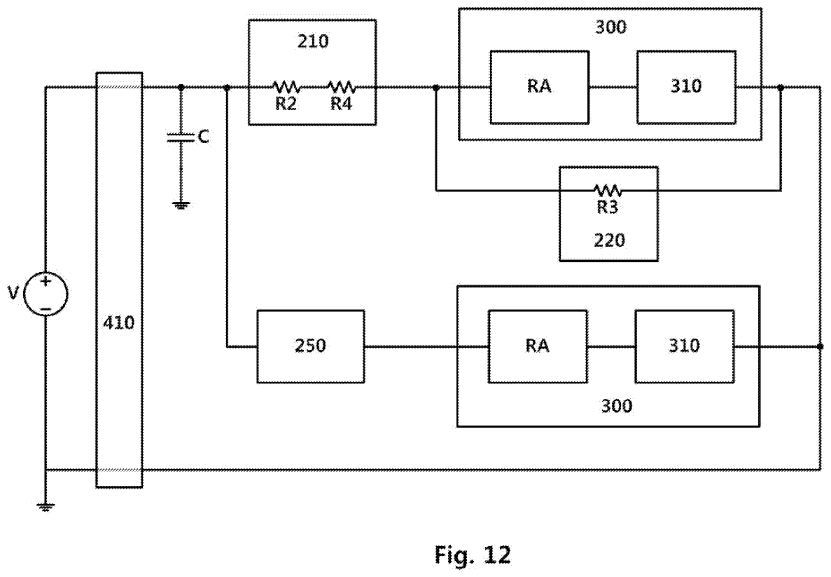

FIG. 12 is a block diagram illustrating operation of the diagnostic strip 10. Referring to FIG. 12, when a body fluid including the target is provided to the entry path 100, the body fluid is provided to the first reaction unit 210 and the second reaction unit 220. An electrical resistance of the first reaction unit 210 reacting with the target is R2, and an electrical resistance of the first reaction unit 210 reacting with the body fluid other than the target is R4. Also, an electrical resistance of the second reaction unit 220 reacting with the body fluid is R3. In the closed loop shown in the drawing, a voltage V is provided from the power providing unit through the connector 410. In an exemplary embodiment, the diagnostic strip 10 further includes a capacitor C which is charged with the voltage provided by the power providing unit.

As described in the above exemplary embodiment, a voltage drop occurring at the resistance R3 is the same as a voltage drop occurring at the resistance R4. Therefore, an electrical characteristic resulting from the reaction unit reacting with the body fluid other than the target may have less influence due to the second reaction unit 220. A voltage component generated by the reaction between the target and the first reaction unit 210 is provided such that the display unit 300 is driven. The provided voltage is divided by a resistor array RA and display devices, and divided voltages are provided to the variable code display unit 310.

FIG. 13 is a schematic diagram showing an overview of the display unit 300. Referring to FIG. 13, the display unit 300 includes the resistor array RA and the variable code display unit 310. In the resistor array RA, a resistor R5 and a unit display device 310e constituting the variable code display unit 310 are connected in series, and these are connected in parallel with a unit display device 310d. A unit display device 310d is connected in series with a register R4, and these are connected in parallel with a unit display device 310c. These are connected in series with a resistor R3, and these are connected in parallel with a unit display device 310b. These are connected in series with a resistor R2, and these are connected in parallel with a unit display device 310a. These are connected in series with a resistor R1.

It is possible to reduce resistance values of the resistor array to form voltage which is divided and provided to unit display devices connected in such a series and parallel structure. For example, all resistance values of the unit display devices 310a, 310b, 310c, and 310d are 100 kΩ, and values of the resistors Rd, Rc, Rb, and Ra are 250 kΩ, 84 kΩ, 41 kΩ, and 25 kΩ, respectively. When a voltage of 5 V is provided to the both ends of the display unit 300, 4 V, 3 V, 2 V, and 1 V are provided to the both ends of the unit display devices 310a, 310b, 310c, and 310d, respectively.

Considering a structure in which different resistors are connected to the respective unit display devices 310a, 310b, 310c, and 310d and all these pairs of a unit display device and a resistor are connected in parallel, resistance values connected in series with the unit display devices 310a, 310b, 310c, and 310d are respectively 45 kΩ, 126 kΩ, 290 kΩ, and 780 kΩ, and the sum of the resistance values is 1241 kΩ. According to the exemplary embodiment shown in FIG. 13 as an example, values of the resistors R4, R3, R2, and R1 are respectively 490 kΩ, 164 kΩ, 81 kΩ, and 450 kΩ, and thus the sum of 780Ω is satisfactory. Therefore, it is possible to reduce an area required for forming resistors.

Referring back to FIG. 12, the diagnostic strip 10 may further include an environmental sensor 250 for measuring an element which has an influence on the reaction between the first reaction unit 210 and the target. According to an exemplary embodiment, the environmental sensor 250 may further include at least one of a hydrogen-ion concentration sensor which is provided with a body fluid including the target and measures a hydrogen-ion concentration (pH) of the body fluid, a temperature sensor which measures a temperature of surroundings of the diagnostic strip 10, and a humidity sensor which measures a temperature of surroundings of the diagnostic strip 10.

According to an exemplary embodiment, the diagnostic strip 10 may display a result measured through the environmental sensor 250 on the display unit 300. Although FIG. 12 shows that a single display unit is connected to the environmental sensor 250, when the environmental sensor 250 includes a plurality of sensors among the hydrogen-ion concentration sensor, the temperature sensor, and the humidity sensor, measurement result values of the sensors may be displayed through a plurality of display units.

Since it is possible to measure a hydrogen-ion concentration of the body fluid and a temperature and a humidity of surroundings of the diagnostic strip 10 by using the environmental sensor 250, a reaction result between the target and the first reaction unit may be calibrated with these factors such that an accurate measurement result can be obtained.

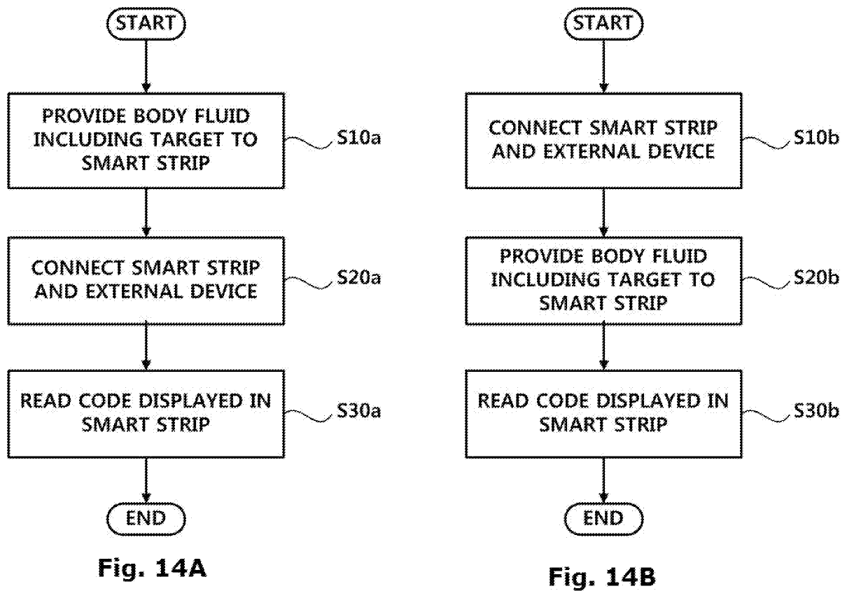

A smart strip and a diagnostic strip using the smart strip will be described below with reference to FIG. 14. FIGS. 14A and 14B are flowcharts illustrating operation of a diagnostic system. FIG. 15 is a diagram showing a state in which the smart strip 10 is connected to a portable terminal that is a power providing unit.

Referring to FIG. 14A, a body fluid including a target is provided to a smart strip (S10A). Since the body fluid and the target are provided, the first reaction unit 210 and the second reaction unit 220 react with the target and the body fluid, and an electrical characteristic thereof is changed.

The connector 410 of the smart strip and a connector of the power providing unit are connected such that power is supplied from the power providing unit (S20A). According to the exemplary embodiment shown in the drawings, the male connector 410 of the smart strip is inserted into a female connector formed in the portable terminal 20, which is the power providing unit, such that the power providing unit and the smart strip are connected. According to an exemplary embodiment not shown in the drawings, female connectors may be formed in both the smart strip and the power providing unit, and the smart strip and the power providing unit may be connected through a cable having male connectors formed at both ends.

Since power is supplied to the smart strip 10, a voltage corresponding to a reaction between the target and the reaction unit is provided to unit display devices included in the variable code display unit 310, and the display unit 300 displays a result corresponding to the reaction (S30A). According to the exemplary embodiment shown in FIG. 15, the display unit 300 displays the reaction result by using a display bar, but may display the reaction result by using a QR code, which is a machine-readable code as shown in the exemplary embodiments of FIGS. 6 and 7.

Also, as shown in the exemplary embodiment of FIG. 14B, the smart strip 10 and the power providing unit may be connected (S10B) such that the capacitor C (see FIG. 12) is charged with a voltage. Subsequently, a body fluid is provided to the smart strip 10 (S20B), and a reaction result may be read (S30B).

Although the present invention has been described with reference to embodiments shown in the drawings to aid in understanding, the embodiments are directed for illustration and merely exemplary. Those of ordinary skill in the art will appreciate that various modifications and other equivalent embodiments are possible from the above embodiments. Therefore, the true technical scope of the present invention should be determined by the accompanying claims.

### INDUSTRIAL APPLICABILITY

Described above.

1. A diagnostic strip for reacting with a target for diagnosis, the diagnostic strip comprising:
a connector configured to be connected to a power providing unit and provide a driving power to the diagnostic strip;
an entry path configured to transfer a body fluid including the target;
a reaction unit configured to react with the target and whose electrical characteristic is changed by the reaction; and
a display unit configured to receive the driving power and whose display state is changed according to the changed electrical characteristic of the reaction unit.

2. The diagnostic strip of claim 1, wherein the connector is any one of a male connector which is inserted into the power providing unit and a female connector into which the connector connected to the power providing unit is inserted.

3. The diagnostic strip of claim 1, wherein the connector is any one of a connector conforming to a universal serial bus (USB) standard and a lightning connector.

4. The diagnostic strip of claim 1, wherein the entry path includes at least one of a capillary for transferring the body fluid to the reaction unit and an exposure unit for exposing the reaction unit so that the body fluid is dropped thereon.

5. The diagnostic strip of claim 1, further comprising an environmental sensor unit configured to detect environmental influence on the reaction between the target and the reaction unit.

6. The diagnostic strip of claim 5, wherein the environmental sensor unit includes at least one of a hydrogen-ion concentration (pH) sensor, a temperature sensor, and a humidity sensor.

7. The diagnostic strip of claim 1, wherein the reaction unit includes a material whose electrical characteristic is changed by an enzyme reaction with the target for detection.

8. The diagnostic strip of claim 7, wherein the reaction unit includes any one of a glucose oxidase whose electrical characteristic is changed by an enzyme reaction with glucose and a cholesterol oxidase whose electrical characteristic is changed by an enzyme reaction with cholesterol.

9. The diagnostic strip of claim 1, wherein the reaction unit includes a material whose electrical characteristic is changed by an antigen-antibody reaction with the target for detection.

10. The diagnostic strip of claim 9, wherein the reaction unit includes any one of an anti-influenza A antibody which reacts with an avian influenza (AI) virus, an anti-epithelial cell adhesion molecule (EpiCAM) antibody, an anti-prostate-specific antigen (PSA) antibody, an anti-human epidermal growth factor receptor 2 (HER2) antibody, an anti-carcinoembryonic antigen (CEA) antibody, and an anti-cancer antigen (CA) antibody which react with cancer cells, and an anti-apolipoprotein B antibody which reacts with lipids in blood.

11. The diagnostic strip of claim 1, wherein the reaction unit includes a probe which is complementarily bound with the target for detection and whose electrical characteristic is changed.

12. The diagnostic strip of claim 11, wherein the reaction unit includes the probe having an aptamer which binds to protein, which is the target for detection, and a nucleotide marker, and nucleotides having a sequence complementary to the target for detection.

13. The diagnostic strip of claim 1, wherein the diagnostic strip detects the target included in animals, human bodies, and food.

14. The diagnostic strip of claim 1, wherein the display unit includes:
a first display whose display state is changed according to the changed electrical characteristic of the reaction unit; and
a second display whose display state is changed according to a detection result of the environmental sensor.

15. The diagnostic strip of claim 1, wherein the display unit includes at least one of an electronic (e)-ink and an electrochromic device.

16. The diagnostic strip of claim 1, wherein the display unit displays a change in the electrical characteristic with any one of a barcode, a quick response (QR) code, and a display bar.

17-30. (canceled)
